(19) United States
(12) Patent Application Publication
(10) Pub. No.: US 2004/0092924 A1
(43) Pub. Date: May 13, 2004

(54) Method and device for follicular hair transplantation

(76) Inventor: Sanjiv A. Vasa, Ahmedabad (IN)

Correspondence Address: Raj S. Dave, Morrison & Foerster LLP, Suite 300, 1650 Tysons Boulevard, McLean, VA, 22102, US

(21) Appl. No.: 10/439,001

(22) Filed: May 16, 2003

(30) Foreign Application Priority Data
May 17, 2002 (IN) 450/MUM/2002

(51) Int. Cl. A61B 18/04

(52) U.S. Cl. 606/32

(57) ABSTRACT

The present invention discloses a transplantation device with a longitudinal groove having a wider slot as a loading area and a narrower slot as a retaining area further developing into a tapered sharpened wedge with depth control performing desired multiple functions of a dissector, site creator, stopper, spiral cavity maker, stretcher, separator and slider capable of harvesting and planting. The device functions without electricity, suction or spring assistance having the advantages of being multifunctional, small, light weight, handy, autoclavable, sterilisable, disposable, having minimum joints and moving parts yet easily manufacturability in wide range of sizes from various materials, with capability to harvest and plant wide range of plant able materials into diverse substrates at enhanced plantation speeds maintaining follicular integrity resulting in better yield. The device of this invention has an optional holder and a handle and is functionally cost effective with easy learning curve and versatile acceptability.

### CROSS-REFERENCE TO RELATED APPLICATIONS

This application claims priority of Indian Patent application no. 450/MUM/2002 filed May 17, 2002, ENTITLED “METHOD AND DEVICE FOR FOLLICULAR HAIR TRANSPLANTATION” by the same inventor. This application is expressly incorporated herein by reference in its entirety.

### TECHNICAL FIELD OF THE INVENTION

The present invention discloses a transplantation device with a longitudinal groove having a wider slot functioning as a loading area extending to a narrower middle portion functioning as a retaining area further developing into a tapered sharpened wedge-shaped end portion with depth control, performing the desired multiple functions of a dissector, site creator, stopper, spiral cavity maker, stretcher, separator and slider capable of harvesting and planting of transplantable materials, maintaining their integrity during transplantation. Further the device has an optional holder and a protective case also capable of functioning as a handle. The device functions without electricity, suction, spring assistance or maintenance and is multifunctional, small, light weight, handy, autoclavable, sterilisable, disposable having minimum joints and moving parts yet can be easily manufactured in wide range of sizes from variety of materials.

### BACKGROUND OF THE INVENTION

The present invention generally relates to the skin and hair related surgery and medical devices for such surgery and processes for hair follicle transplantation. It is found that now-a-days hair loss of an individual can be undesirable and, therefore, there are various methods and processes used to overcome such problem, such as hair pieces, topical application, oral medication, scalp reduction, scalp flaps and hair transplantation. But the patients are looking for something that looks natural, long lasting or permanent, affordable, without side effect and with low or no maintenance.

The present invention of follicular hair transplantation is now widely accepted best hair replacement alternative as it fulfills most of the above criteria. Clinical study has revealed that when longer lasting hair from back and side of scalp are transplanted over the area of baldness, hair continues to stay viable and grow.

Traditional transplantation of large punches became unacceptable due to its poor cosmetic result. It also produced loss of valuable hair grafts during and after transplantation procedure. This led to the preparation of mini, micro and ultimate follicular hair grafts and transplanting them into desired area [Marritt E. Siongle hair transplantation for hairline refinement: A practical solution. J. Dermatol. Surg. Oncol. 10:962,1984.; Bradshaw W: Quarter grafts: A technique for minigrafts. In Unger WP, Nordstorm REA(eds): Hair transplantation .Ed 2, New York, Marcel Dekker. 1988.; Bernstein RM Rassman WR. “The Logic of follicular unit transplantation” Dermatologic Clinics 1999; 17 (2): 277-295.].

The actual placement of hair follicle is the most difficult and important part of hair transplantation on which the ultimate result depends. Most natural result of hair transplantation demands meticulous harvesting of the transplantable material from the donor site and plantation of several hundreds of grafts in short time without damaging the roots. Several methods and instruments have been developed based on steps that involve damage free harvesting of the transplantable material from the donor site, creation of appropriate recipient site, keeping the site open during the plantation process, placing the graft into the site, maintaining its integrity and ensuring closure of the site after the plantation process is completed.

In the case of most devices described in the prior art, harvesting as well as plantation is not possible with the same instrument.

Several methods and instruments are known in prior art to perform harvesting and plantation separately.

Harvesting of transplantable material are divided into “open donor” and “closed donor” methods.

Open donor methods are reported by Sasagawa M. (Hair transplatation Japan J Dermatology 1930; 30;493); Okuda S. (Clinical and experimental studies of transplantation of living hair. Japan J. Dermatology 1939: 46; 135-8); Orentreich N. (Autografts in alopecias and other selected dermatological conditions; Ann. New York Academy Sciences, 1959; 83; 463-79). Generally 4 mm diameter punches were used to harvest hair bearing skin grafts. The open wounds were allowed to heal by fibrosis resulting in scars, which further altered the direction of the hair making subsequent harvesting even more difficult. Division of large punches into smaller split grafts or quarter grafts continued the further transaction of hair, which gave unacceptable cosmetic results. Harvesting through the smaller diameter punches produced better cosmetic results but technically made it more difficult as the smallest change in angle during harvesting transected the hair follicle.

James Arnold (Int. J. of aesthetic and restorative surg. Vol.3 number 2. 1995 148-153) describes closed donor methods, where harvesting through multibladed knife generates mini and micro grafts superior to the small punch grafts but carries significant amount of transaction. The issue is that sutures are used to close the donor area, which leaves scars. Limmer B. L. (Dermatology Syrg. 1994; 20:789-93.) discloses the use of single strip harvesting and then cutting into thin slices (slivers) under stereoscope to produce maximum yield and minimum damage producing most acceptable results except that this also suffers from the problems of resultant scars.

To avoid the production of linear scars Dr. Rassman (Dermatology Surgery 2002; 28: 720-728) suggested again the open donor method of Follicular Unit Extraction directly from the donor site by minimal invasive surgery based on a publication by Dr. Inaba M. (Andrgenetic Alopcia; Modern concept of pathogenesis and treatment. Tokyo; Springer-Verlag 1996, 238-45). This helps in correcting the limited small area of baldness in selected suitable cases but suffers from the shortcoming that it takes longer time compared to single strip harvesting methods.

Methods involving manually or electrically driven rotating sharp edge small punches into the skin are time consuming and essentially blind techniques with possibilities of damaging the follicles by lateral heat generated by high-speed rotation of punches and clogging of punches by the harvested plugs making the unloading operation difficult and damaging. such methods may cause further damage to deeper structures due to lack of depth control.

The multiple strips or single strip harvesting methods, are known to cause damage due to the follicular transaction (even working through high magnification) and resulting in scars at the donor sites.

Small diameter punches used for direct follicular extraction from the donor site are not suitable for multi hair follicular units as they lack depth control and ability to change direction during superficial to deep dissection to maintain alignment parallel to the splayed out follicles.

Also, harvesting as well as plantation is not possible with a common instrument as described in prior art.

The plantation process generally involves steps that include creation of an ideal recipient site, keeping the site open and placement of the graft into the site maintaining its integrity.

In one of the methods, the recipient site is created by removing skin with round or linear punches that operates manually or by electrical force. U.S. Pat. Nos. 5,895,403, U.S. Pat. No. 5,922,000, U.S. Pat. No. 5,792,163, U.S. Pat. No. 5,611,810, U.S. Pat. No. 5,693,064 describe the methods based on the punch technique. These methods and instruments suffer from the drawbacks of destroying blood vessels and collagen weakening the elastic support. This also results in more bleeding, increased coagulum, reduced perfusion and delayed healing. The operation required regular cleaning to remove clumps of tissue blocking the instrument and invariably required prolonged pressure to maintain the graft in place to prevent popping out. Further, this led to enhanced crust formation, delayed hair care and is cosmetically unacceptable end result.

Techniques based on laser and radio frequency waves to create sites destroy body tissue by lateral heat. In addition to excessive bleeding, the lateral thermal damage of the sidewalls of cavity with narrow base of the cavity increase pop-out ratio. It also has the undesirable feature of producing peculiar sounds and odor produced due to tissue burning. [Bernstein RM, Rassman WR: Laser hair transplantation: Is it really a state of art? Lasers in Surgery and Medicine 1996; 19: 233-235]

The standard surgical processes followed may be classified under those creating “slits”, micro holes, dilation to keep the sites open followed by the placement of grafts into them and letting the grafted hair to stabilize in its transplanted site.

Several methods are in use for placement of hair grafts maintaining its integrity. Placement can be performed separately or simultaneously with the creation of recipient sites. In one method after creating site, graft is picked and held by forceps or similar grasping instrument and pushed into the recipient slit or hole. In addition to drying of tissues, mechanical damage to the vital cells is possible during holding, grabbing, pinching, dragging and pushing. Trying to insert graft into cavity can also produce crushing, squeezing, folding, distorting, jamming, distracting and bending of hair grafts. In another method first the instrument is loaded with graft and then using the instrument graft is inserted into the recipient site. Some have described plurality of grafts in one instrument.

In case of multiple grafts, loaded in one instrument, the grafts stick to each other so much that they are difficult to separate and during maneuver they may fall out from the instrument. Within the instrument it is difficult to maintain them moist and cool. Keeping them in saline, they move or even fall out from the pre-loaded instrument. During plantation planting surface gets flooded with stored saline in the tube obstructing the view and delaying plantation. As longer tubes are required to store more grafts, maneuvering of instrument and handling of grafts become difficult.

In case of slits, cold steel has been found to produce minimum damage by distracting tissue. Blades (number 11, 15) and trokars of different shape (triangular, mercedes, rectangular) are used to create slits in the skin. (Arnold James, Mini blades and mini blade handle for hair transplantation American journal of Cosmetic Surgery Vol. 14 no. 2 1997 195-200.) In this method, the step of “slits creation” is an additional step in the process and at the same time increases the burden of counting and isolating grafts to match the number, size including width and depth of the slits. It is possible to miss sites, piggy backing (more than one graft in one cavity), hair under graft, slipping too deep and rough placing with multiple attempts. These consume additional time of the surgeon and impact the cost of the surgery.

Thus, there is a need for a simple instrument capable of providing non-traumatic hair transplant, at the same time maintain follicular integrity, provide better transplantation results with minimal scar and bleeding on the substrate and is less strenuous on the operator.

### SUMMARY OF THE INVENTION

An embodiment of the invention is to provide a functionally cost effective manually operated instrument that is capable of harvesting as well as planting wide range of transplantable materials into diverse substrates at enhanced plantation speeds maintaining follicular integrity resulting in better yield.

According to another aspect of the invention there is provided an instrument for silent non-traumatic hair transplantation that ensures feather-touch non-traumatic method of picking up the hair follicle only by its extra cuticle part of hair from the cool isotonic solution and inserting them in the loading area of suitable size in a device avoiding the drawbacks of grabbing, holding, pinching, dragging, pushing and drying.

According to another aspect of the invention there is provided a disposable instrument that has capacity to harvest the planting material under direct vision in-vivo and in-vitro with easy unloading and depth control maintaining follicular integrity.

According to another aspect of the invention there is provided a disposable instrument for the hair transplantation, which is of convenient size, compact, modular with multifunctional non-moving components, functioning without electricity, suction, maintenance and spring assistance, manufactured in varying sizes from variable materials to accommodate different size follicles, that can be sterilized, stored and easily maneuvered by the surgeon.

According to another aspect of the invention there is provided an instrument for hair transplantation capable of site creation to provide a consistent open orifice with separated walls of a spiral cavity on the substrate of width and length that is appropriate to snugly fit the follicle, adjustable to plant the follicle at a desired angle and direction to the substrate, controlling the transplant density of the substrate with minimum structural damage to the substrate and producing minimum bleeding and simultaneously maintaining maximum blood supply to produce optimized yields.

According to another aspect of the invention there is provided an instrument capable of hair transplantation by a surgeon over a wide range of substrates including scalp, skin having eyebrow, beard, mustache, eyelash or any other desired area.

According to another aspect of the invention there is provided an instrument capable of hair transplantation to handle any kind of soft or coarse hair.

According to another aspect of the invention there is provided an instrument for hair transplantation by preserving the graft integrity during harvesting, transfer, loading and plantations.

According to another aspect of the invention there is provided an instrument for hair transplant, which ensures protection to the already transplanted hair or existing hair on the substrate during transplantation process.

According to another aspect of the invention there is provided an instrument for hair transplant using which plantation can be performed in one rapid single motion technique, eliminating second and subsequent maneuvers.

According to another aspect of the invention there is provided an instrument for hair transplant capable of keeping the orifice in a stretched open condition and walls of the cavity separate during the transplantation process to enable easy insertion of follicle.

According to another aspect of the invention there is provided an instrument for hair transplant capable of insertion of follicle under direct vision avoiding rough placing with multiple attempts, popping out, drying, crushing, squeezing, folding, distorting, destructing, jamming, missing sites, piggy backing (more than one graft in one cavity), hair under graft, bending, pitting (slipping too deep) or damaging the neighboring follicles in any manner.

According to another aspect of the invention there is provided an instrument for hair transplant, which performs plantation under direct control with ability to correct, change or revert the angle, direction and depth under direct vision.

According to another aspect of the invention there is provided an instrument for hair transplant, which does not create air lock and backpressure during plantation.

According to another aspect of the invention there is provided an instrument for hair transplant capable of transplanting whole or part of the follicle.

According to another aspect of the invention there is provided an instrument for hair transplant capable of planting tissues with or without the associated fiber or hair.

According to another aspect of the invention there is provided an instrument for hair transplant where loading of the hair is possible from the top without causing damage to the follicle by sharp edges and traction.

According to another aspect of the invention there is provided an instrument for hair transplant capable of transplanting artificial hair.

The present invention is a single instrument capable of loading the follicle of transplant material onto it for transplantation, creating a recipient site on a substrate, keeping the site open, and planting the follicle of the transplant material into the substrate using the same instrument.

Some of the advantages are use of minimum force with easy maneuvering during the procedure. The small muscles of hand experience very minimal fatigue. The transplants are performed faster in lesser time with better results. Learning curve is simple and fast for novice. Preservation of graft integrity during transfer, loading and plantation as well as free handling instrument without electricity, spring device, suction tubing or other attachments makes this instrument ideal choice for transplant. Change of part is not required during procedure; cleaning and removal of plugs during plantation are not required; the instrument is able to handle any kind of soft or coarse hair. Lateral thermal damage of substrate is avoided. Holding and supporting the grafts is very well achieved that there is no “flying” or “falling off” of the grafts from instrument. There is no need for additional measuring device as grafts are visually judged to fit into the loading area of transplanter. Application of force in a straight line along the long axis of instrument is permitted. Artificial hair planting is also allowed. The instrument is good enough even for chubbier grafts which require less trimming. Less time is wasted in arranging, separating and counting of follicle with creating, matching and counting recipient sites. Minimum spacing between adjacent grafts gives increased density with more natural appearance. Reduced operative time leading to low physician cost allowing physician to engage in other activities

Other major advantages of this invention are that it allows performer (operator) of this procedure to be in total control of positioning and spacing grafts concentrating fine points of insertion like site, angle and direction. Existing hair are not wiped off or trapped or entangled during insertion. More than one operator is allowed to transplant on the same patient at the same time. Minimal to no dead space is left around planted hair follicle. This assures maximum contact of graft with surrounding tissue so that oxygenation can be quickly reestablished. By eliminating dead space there's less coagulum formed and wound healing is facilitated with minimum scarring. The grafts are easily adjusted after plantations for minor adjustment.

The instrument has also an added advantage of being small, light weight, convenient (comfortable) length, without use of electricity, suction and spring assistance, with minimum joints or moving parts, disposable (to avoid cross infection), available in varying size to accommodate different size follicles, affordable, quiet operation (without noise pollution), longer life span of disposable allowing more follicle plantation with only one transplanter not requiring any maintenance.

Further embodiments and advantages will become apparent from a consideration of the ensuing description and drawings.

### DETAILED DESCRIPTION OF THE FIGURES

Reference numerals in drawings:

1. Handle/Case

2. Holder/Hub

3. Stopper

4. Transplanter

5. Loading Area

6. Retaining Area

7. Wedge shaped end

8. Hair

9. Skin

Definitions:

A “transplant material” is a solid or semisolid material like fiber and its alike, living or non-living, but not a liquid that could fall out from the transplanter under gravity through the openings of the transplanter.

The term “harvesting” means up-rooting a product from the substrate preferably without damaging the product.

A “stopper” is a device or body that can restrict a depth to which the transplanter can pierce the substrate.

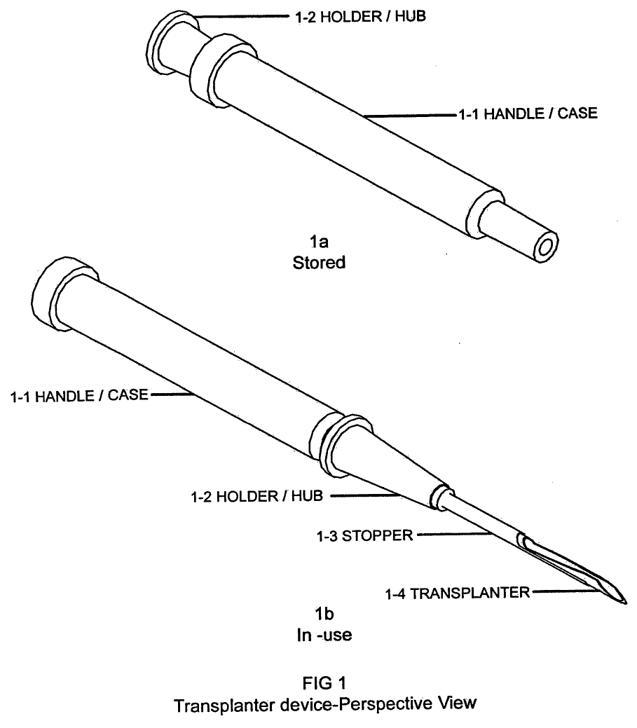

FIG. 1 is a perspective front view illustrating transplanter device, which is a conventional transplanter device in a stored position (FIG. 1a). FIG. 1b shows the perspective front view illustrating transplanter device in-use position with handle/case of the transplanter device referenced as 1-1, holder/hub referenced as 1-2, stopper referenced as 1-3 and the transplanter as 1-4.

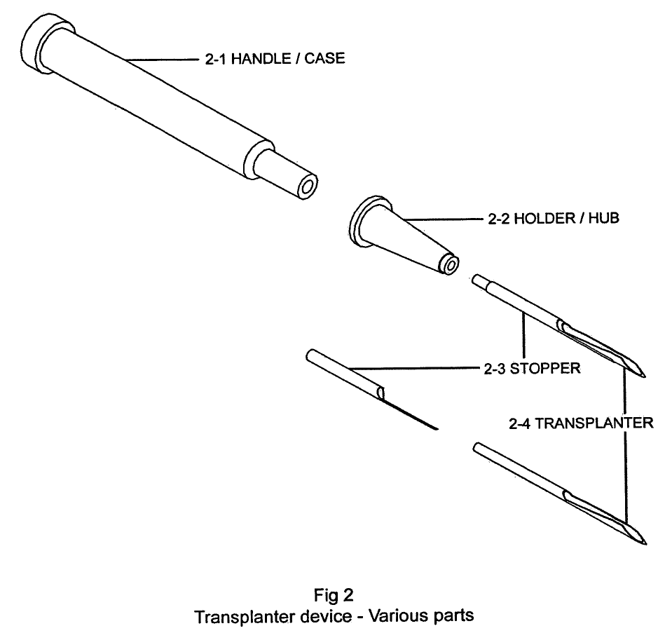

FIG. 2 shows the perspective view of the individual parts that make up the transplanter device—2-1 handle/case, 2-2 holder/hub, 2-3 stopper and 2-4 transplanter.

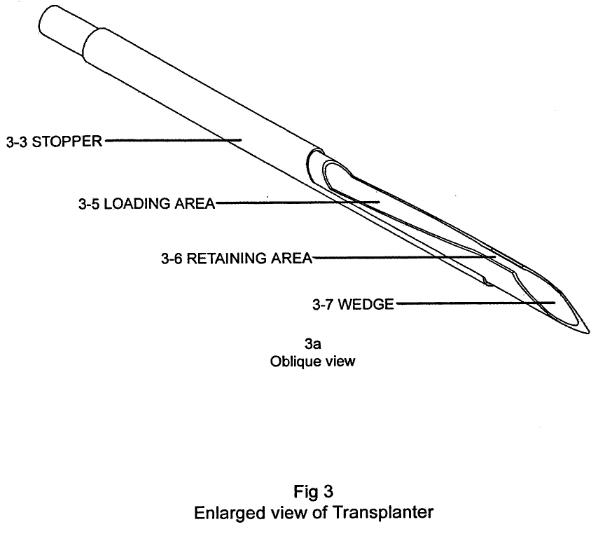

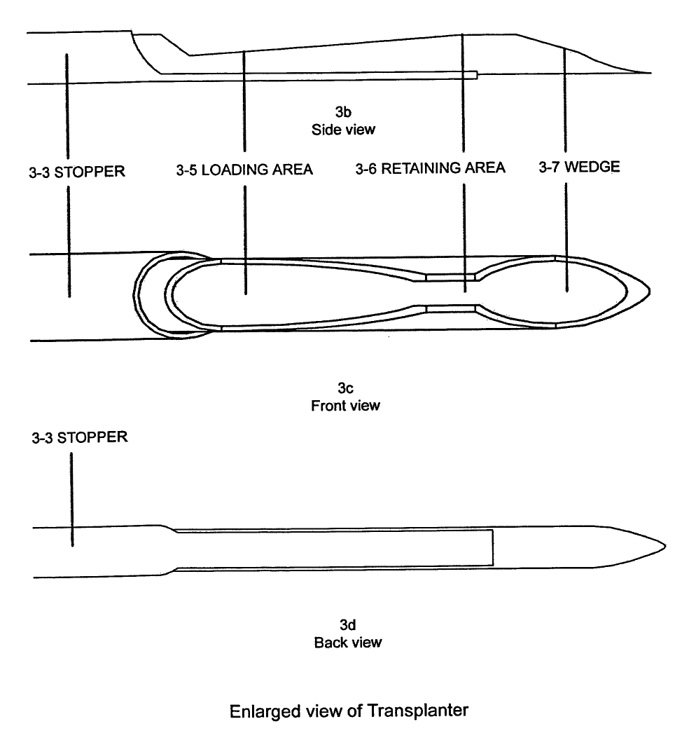

FIG. 3 shows the enlarged view of the transplanter with FIG. 3a depicting the oblique view FIG. 3b the side view, FIG. 3c the front view and FIG. 3d the back view of the transplanter device.

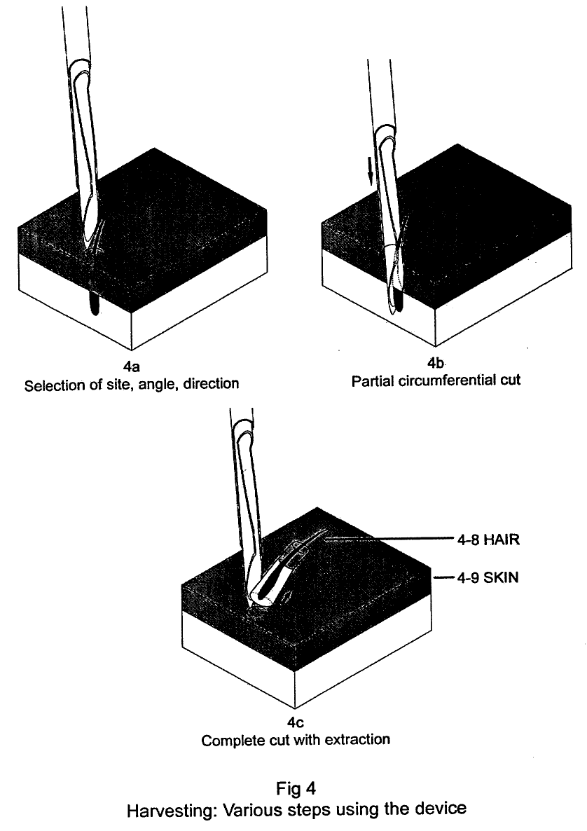

FIG. 4 is schematic view depicting the harvesting steps of transplantation. FIG. 4a illustrates the selection of site, angle and direction on the substrate with the depth/height that is needed to harvest. FIG. 4b the partial circumferential cut and FIG. 4c the complete cut with extraction of hair 4-8 from the substrate-skin (4-9).

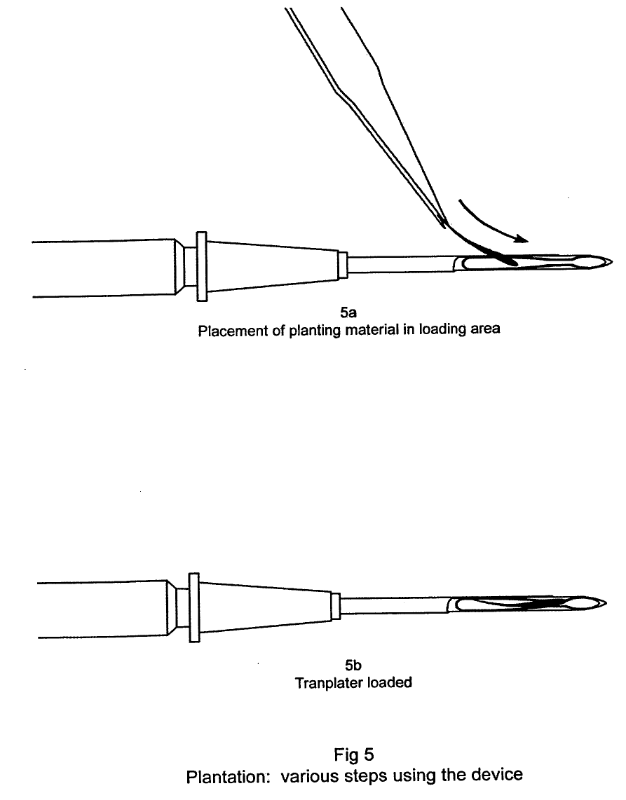

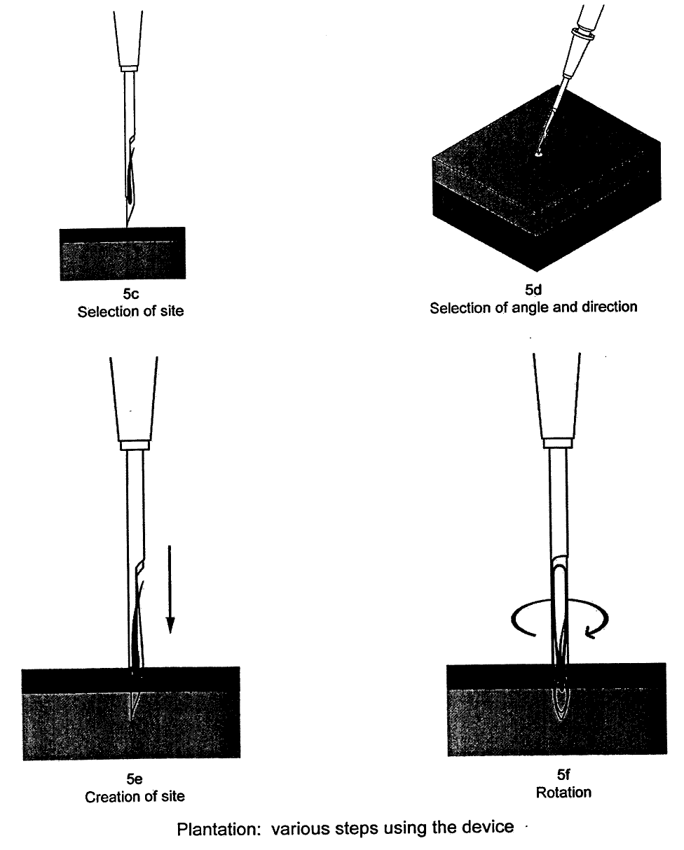

FIG. 5 shows the steps involved in planting the transplantable material with FIG. 5a showing the placement of planting material in the loading area of the device, FIG. 5b the top view of the planter with follicle loaded into the transplanter device, FIG. 5c further depicts the selection of the site, FIG. 5d depicts the selection of angle and direction at which the transplanter device is to enter the substrate during transplantation, FIG. 5e shows the creation of the transplantation site on the substrate, FIG. 5f the rotation of the device for clearer view of the follicle before the transplantation, FIG. 5g shows the sliding of the material with the help of a sliding instrument, FIG. 5h the complete insertion of the material into the substrate from the device, FIG. 5i the withdrawal of the device with the sliding instrument holding the transplant material in place on the site of the substrate and FIG. 5j the completed transplantation.

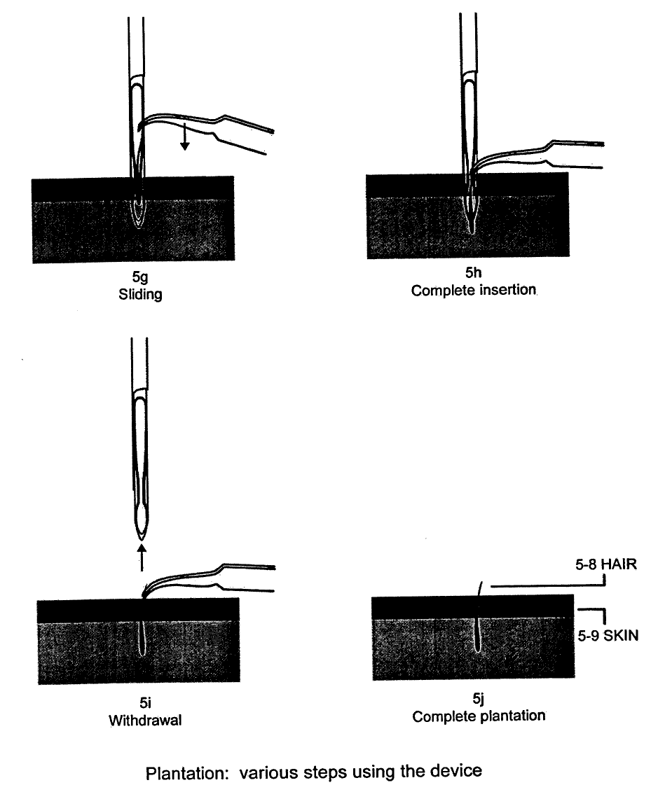

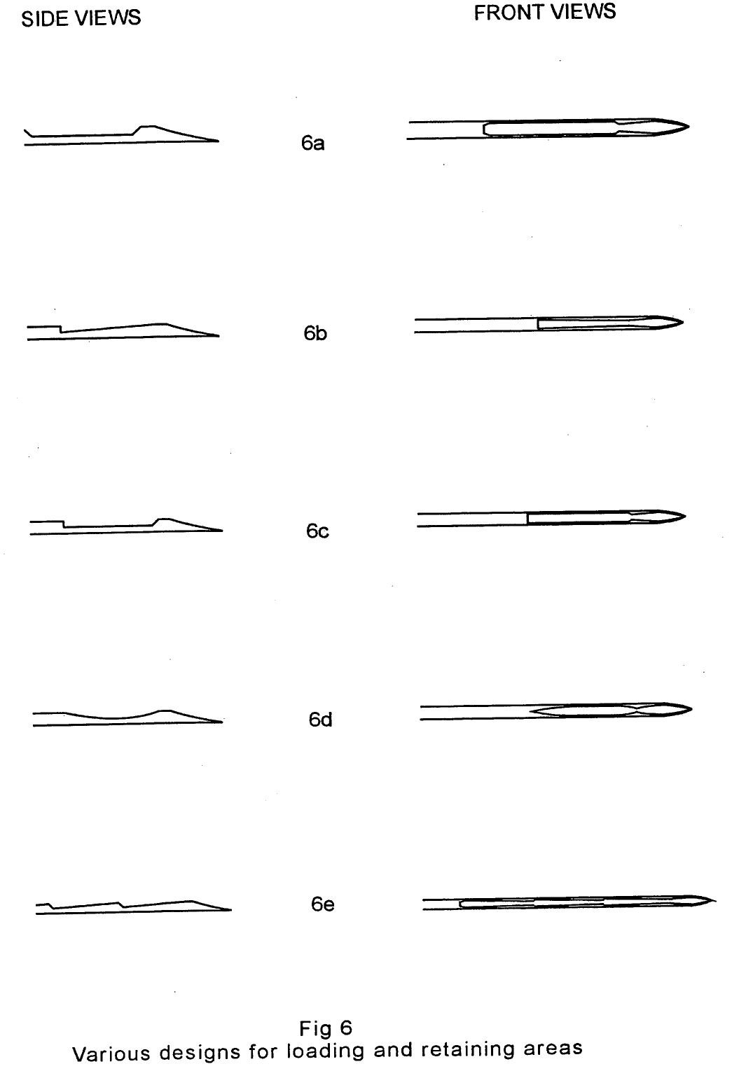

FIG. 6 shows the side views and front views of the various designs of loading area and retaining area of the transplanter device.

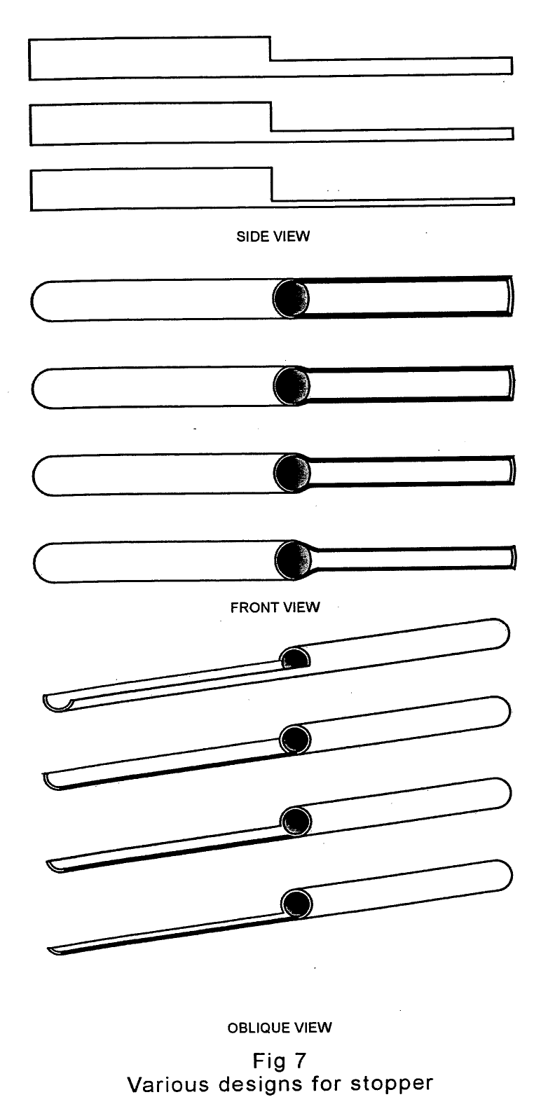

FIG. 7 shows the front, side and oblique views of various designs for the stopper.

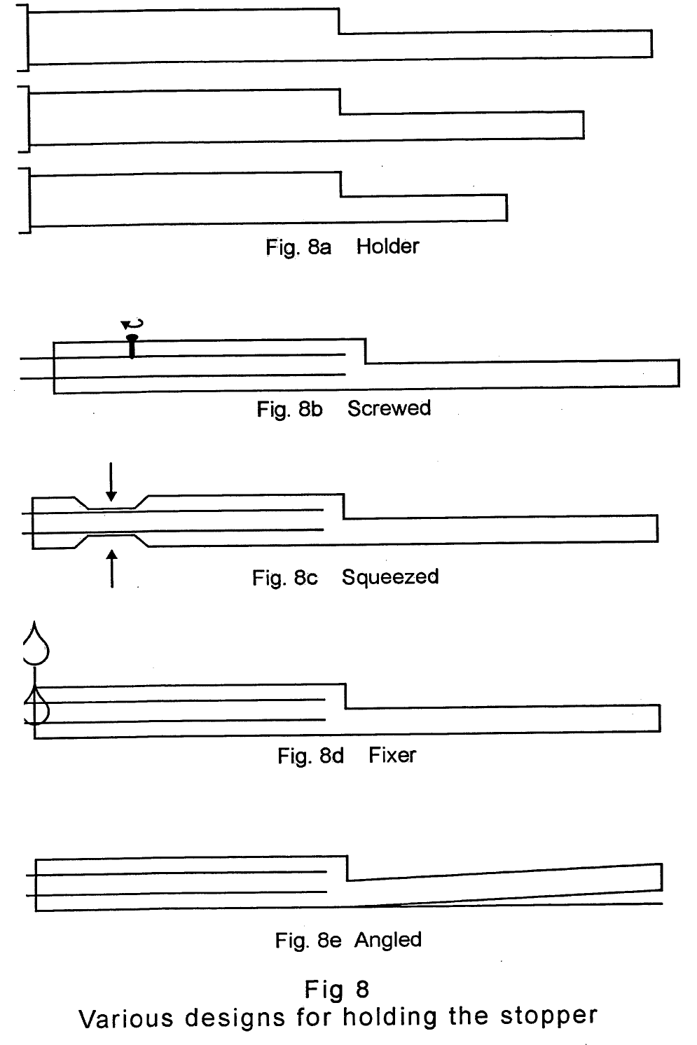

FIG. 8 shows the various designs for holding or connecting the stopper to the transplanter device. FIG. 8a—the stopper flange length adjusted as per required length of hair follicle and/or the having a holder attachment at different levels, FIG. 8b—the stopper length adjusted and fixed with a small screw device, FIG. 8c—the stopper may be squeezed tight over transplanter, FIG. 8d—the stopper is fixed with fixer, FIG. 8e—the stopper flange may be angled to the body for fixation

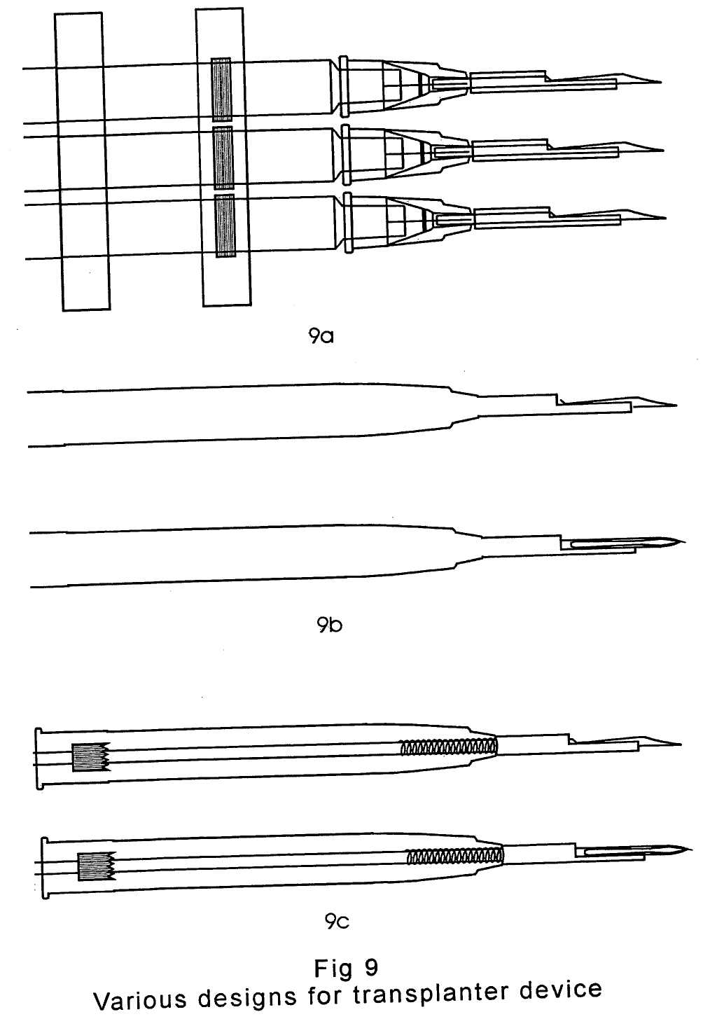

FIG. 9 shows the various designs for the transplanter device with FIG. 9a showing plurality of transplanter arranged side by side and held together by a common member, FIG. 9b the transplanter device where handle, holder, transplanter and stopper are made in one piece and not detachable and FIG. 9c is schematic detailed view of a transplanter device where various parts are movable and detachable.

### DETAILED DESCRIPTION OF THE PRESENT INVENTION

The present invention relates to a device used to harvest transplantable material from donor sites and plant the same material such as hair and its like on diverse substrates comprising a solid and/or hollow elongated member (transplanter) of an appropriate length, width, wall thickness and shape, longitudinally developing into a slotted groove having a wider slot as a top loading area extending to a narrower slot as a middle retaining area further developing into a tapered wedge shaped end. The wedge shaped end is of appropriate length, width and shape with sharpened edges to enable piercing a substrate for harvesting and/or planting to appropriate depth determined and controlled by a stopper located along the surface of the transplanter keeping top loading area, middle retaining area and wedge shaped end open. The transplanter has optional holes of appropriate number, size, shape, at appropriate positions along the transplanter to release air locked during transplantation.

The transplanter has an optional holder [hub] structured at the holder's [hub] lower end to enable connection with the stopper and/or the transplanter (FIGS. 1b, 2). The upper end of the holder [hub] is appropriately structured to fit an optional handle [case] (FIGS. 1b, 2).

The transplanter has an optional handle with an elongated body, a front end and a rear end. The optional handle is so structured at the front end to enable housing the holder [hub] and/or the transplanter during use (FIGS. 1b, 2) while the rear end is so structured to accommodate the holder [hub] and/or the transplanter when placed inside the handle to function as a case for storage during non-use (FIG. 1a).

The slotted groove described above is broader at the top loading area with gradual narrowing towards the middle retaining area opening into the tapered wedge shaped end.

The width of the groove at top loading area is about more than one to about five times the diameter of the planting material to facilitate the containment of the planting material, ensuring its smooth loading, unloading and movement along the groove for subsequent operations.

The width of the groove at middle retaining area is such that it does not allow the planting substance to pop out from the groove but at the same time allows easy passage of the transplantable material to and from the top loading area to the tapered wedge shaped end.

The edges of the wedge shaped end are sharp and tapered in a manner to allow easy passage of transplanter into the substrate with rotation. The width and shape of the wedge shaped piercing end is so designed that during harvesting it enables creation of circumferential cut around the hair follicle and during plantation it enables the creation of an appropriate site for the transplanting operation by maintaining the opening of the cavity stretched and walls of the cavity dilated/separated to facilitate easy insertion of the transplantable material into the created site with the help of a sliding device.

A slotted arrangement along the outer surface of the transplanter at a predetermined distance from its lower wedge shaped end also functions as a stopper, which restricts the depth to which the transplanter may pierce the substrate on which the transplanting operation is to be performed. In one of the embodiments of the invention, the stopper arrangement may be created as an integral part of the transplanter or optionally along the common outer surface of the top loading area and/or the middle retaining area of the transplanter. In another embodiment, the stopper may be constructed by sleeving the inside of the transplanter with inter-fitting surfaces, in such a manner that it does not cover the top loading area, middle retaining area and the wedge shaped end, with an appropriate “discontinuity ” created at a predetermined position from the lower end along the surface of the transplanter.

A holder [hub] is shaped in a manner that its lower end is capable of being attached and/or fixed to the stopper and/or the transplanter at the stopper end and its upper end structured to fit at either ends of the an optional handle [case].

An optional handle [case] of appropriate length and shape with a front end and a rear end, is so structured to enable attachment of the holder [hub] and/or transplanter with or without stopper at its front end for easy maneuvering during the harvesting and/or planting operation. In one embodiment of the invention the handle [case] may be structured hollow with dimensions to house the holder (hub) and/or transplanter with or without stopper in it for storage when transplanter is not in use (FIG. 1a). The integrated self-contained system of the transplanter, stopper, holder [hub] and handle [case] may be sterilized together and packed for operational convenience.

The device is capable of harvesting transplantable material from a donor site as well as plantation of the transplantable material in the suitable substrate performing the desired multiple functions of a dissector, site creator, stopper, spiral cavity maker, stretcher, separator and slider maintaining their integrity during transplantation.

Further the device is protected in a case (handle), which can be used as a handle during transplantation.

For follicular extraction, the transplanter with holder [hub] is taken out from the case (handle) and attached to the lower end of the case (handle), which then functions as a handle (FIG. 1b). The site, angle and direction are selected (FIG. 4a). Then, the wedge shaped end is inserted into the skin close to the emerging hair but not totally encircling it and then advancing into the deeper tissue (FIG. 4b) up to a desired depth obliquely and away but parallel to the presumed direction of the hair preserving splayed out hair roots.

The transplanter is then pulled out and reinserted in the same fashion into the skin repeatedly to create a complete cut encircling the hair follicle from superficial to deep (FIG. 4c).

The follicle is pulled out gradually with minimum traction force by holding it from the collar of the skin or extra cuticular part of the hair. Extra strokes may be required during extraction to free the follicle completely if found attached with deeper tissue at some point.

During harvesting, if the follicle is jammed or clogged in the transplanter, it is unloaded through the loading area holding it with the extra cuticular part of the hair or kept moist and cool for plantation into the recipient site.

During planting, the transplanter with holder [hub] attached to the lower end of the handle [case] is loaded with the follicle through the wider end of the top loading area (FIG. 5a) in such a way that the lower end or bulb of the follicle lies in the middle retaining area of the transplanter towards the wedge shaped end (FIG. 5b).

The site of transplantation is selected on the substrate (FIG. 5c) where transplantation is to be made, the desired incision, angle and direction are determined (FIG. 5d) and then the device is advanced up to desired depth on the substrate to create the incision (FIG. 5e) with rotation (FIG. 5f). Rotation ends in such a way that the slotted groove faces the operator for direct visualization of the follicle.

Downward movement of an external sliding instrument sited in the groove (FIG. 5g) slides the follicle into the cavity to complete insertion (FIG. 5h).

As the follicle reaches the bottom of the cavity, the transplanter is pulled out keeping the follicle in place by the sliding device (FIGS. 5i and 5j).

This device functions without electricity, suction or spring assistance when used for transplantation. The instrument has the advantage of being multifunctional, small, light weight, handy, autoclavable, sterilisable, disposable, having minimum joints and moving parts yet easily manufacturability from various material in wide range of sizes, with capability to perform harvesting as well as plantation of wide range of transplantable materials into diverse substrates at enhanced plantation speeds maintaining follicular integrity resulting in better yield. The device of this invention is functionally cost effective with easy learning curve and versatile acceptability.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

This application discloses several numerical range limitations. The numerical ranges disclosed inherently support any range within the disclosed numerical ranges even though a precise range limitation is not stated verbatim in the specification because this invention can be practiced throughout the disclosed numerical ranges. Finally, the entire disclosure of the patents and publications referred in this application are hereby incorporated herein by reference.

What is claimed is:

1. A transplanter device comprising:
at least one elongated member comprising a wedge shaped end, said wedge shaped end having sharpened edges sufficient to penetrate and cut a substrate,
wherein said transplanter device is capable of acquiring a transplant material from said substrate and releasing said transplant material to said substrate.

2. The transplanter device of claim 1, further comprising at least one stopper member with a stopper end, said stopper member operatively connected to said elongated member to prevent said elongated member from penetrating said substrate beyond a predetermined depth in said substrate.

3. The transplanter device of claim 2, wherein said elongated member further comprises a middle retaining area and a channel formed in said middle retaining area and extending through said wedge shaped end, said middle retaining area being adjacent to said wedge shaped end, said channel having a size sufficient to slide and position said transplant material, and said middle retaining area having an opening to provide visibility and access to contents in said middle retaining area.

4. The transplanter device of claim 3, wherein said elongated member further comprises a top loading area, said channel extending through said top loading area, and said top loading area having an opening to provide access to said channel to receive said transplant material into said channel.

5. The transplanter device of claim 4, wherein a width of said channel is between about 1-5 times a width of said transplant material.

6. The transplanter device of claim 1, wherein said substrate comprises an area of a human body selected from the group consisting of scalp, skin having eyebrow, beard, mustache, and eyelash.

7. The transplanter device of claim 1, wherein said transplant material is a natural hair or an artificial hair.

8. The transplanter device of claim 1, wherein said elongated member further comprises at least one hole along a body of said elongated channel, said hole allowing the release of air locked during said transplant.

9. The transplanter device of claim 1, wherein said elongated member has a plurality of holes along a body of said elongated channel, said plurality of holes allowing the release of air locked during said transplant.

10. The transplanter device of claim 1, wherein said elongated member further comprises a plurality of raised slots arranged along an outer surface of said elongated member, said plurality of raised slots being equidistant from said wedge shaped end, said plurality of raised slots preventing said transplanter device from being inserted into said substrate beyond said raised slots.

11. The transplanter device of claim 2, wherein said stopper member is a sleeve, said sleeve being slid able over said wedge shaped end of said elongated member, and said sleeve being positioned to allow access to a top loading area, said middle retaining area and said wedge shaped end of said elongated member.

12. The transplanter device of claim 2, wherein said stopper member is integrated into the surface of said elongated member, said stopper member prevents insertion of said transplanter device beyond said stopper member, and said stopper member is positioned to allow access to a top loading area, said middle retaining area and said wedge shaped end of said elongated member.

13. The transplanter device of claim 2, wherein said stopper is an adjustable stopper, said adjustable stopper constructed by sleeving said elongated member with an inter-fitting surface, and said adjustable stopper being positioned to allow access to a top loading area, said middle retaining area and said wedge shaped end of said elongated member.

14. The transplanter device of claim 2, wherein said stopper is an attachable stopper, said attachable stopper is constructed by sleeving said elongated member with inter-fitting surface, said attachable stopper positioned to allow access to a top loading area, said middle retaining area and said wedge shaped end of said elongated member.

15. The transplanter device of claim 1, wherein said transplanter device further comprises a holder, said holder rotatably connected at one end of said holder to an end opposite to said stopper member of said transplanter device, said holder providing easy maneuvering of said transplanter device during said transplant.

16. The transplanter device of claim 1, wherein said transplanter device further comprises a long hollow case, said case having a front end and a rear end, said front end of said case being rotatably connected to said transplanter device at end opposite to said stopper member, a length of said case being of a predetermined length to house said transplanter device.

17. The transplanter device of claim 15, wherein said transplanter device further comprises a long hollow case, said case having a front end and a rear end, said front end of said case being rotatably connected to said holder at an end opposite to the end connected to said stopper member, a length of said case being of a predetermined length to house said transplanter device and said holder.

18. The transplanter device of claim 16, wherein said transplanter device, said stopper, said hollow case are made of a sterilisable material.

19. The transplanter device of claim 16, wherein said transplanter device, said stopper, said hollow case are made of a disposable material.

20. The transplanter device of claim 17, wherein said transplanter device, said stopper, said holder, said hollow case are made of a sterilisable material.

21. The transplanter device of claim 17, wherein said transplanter device, said stopper, said holder, said hollow case are made of a disposable material.

22. The transplanter device of claim 1, wherein said stopper is solid.

23. The transplanter device of claim 1, wherein said stopper is hollow.

24. The transplanter device of claim 2, comprising:
a plurality of said elongated members;
a plurality of said stopper members, said plurality of stopper members being connected to said elongated members; and
further comprising a holding member operatively connected to said stopper members, said holding member capable of rotating said elongated members independently and collectively about an axis.

25. The transplanter device of claim 24, wherein said holding member further comprises a handle to hold said transplanter device.

26. A method to harvest a transplant material comprising:
providing a transplanter device of claim 1; and
harvesting said transplant material from the substrate.

27. The method of claim 26, further comprising selecting a site, angle, direction on said substrate for harvesting said transplant material.

28. The method of claim 27, further comprising inserting and rotating said transplanter device in said substrate.

29. The method of claim 28, further comprising releasing a root of said transplant material from said substrate.

30. The method of claim 29, further comprising withdrawing said transplanter and the transplant material from said substrate.

31. A method to plant a transplant material, comprising:
providing a transplanter device of claim 1; and
embedding said transplant material into said substrate.

32. The method of claim 31, further comprising loading said transplanter device with said transplant material, wherein a root of said transplant material lies towards said wedge shaped end of said transplanter device and a body of said transplant material lies in said middle retaining area.

33. The method of claim 32, further comprising positioning said wedge shaped end of said transplanter device at a desired transplant site, angle, direction on said substrate.

34. The method of claim 33, further comprising inserting said transplanter device into said substrate to form a spiral cavity.

35. The method of claim 34, further comprising rotating said transplanter device in said cavity to allow direct view of said transplant material in said elongated member.

36. The method of claim 35, further comprising providing a sliding instrument to access said transplant material in said elongated member.

37. The method of claim 36, further comprising sliding said transplant material from said transplanter device into said cavity with said sliding instrument.

38. The method of claim 37, further comprising withdrawing said transplanter device from said cavity.
